(19) United States
(12) Patent Application Publication
(10) Pub. No.: US 2019/0091218 A1
(43) Pub. Date: Mar. 28, 2019

(54) LEVOCETIRIZINE AND MONTELUKAST IN THE TREATMENT OF RADIATION-MEDIATED CONDITIONS

(71) Applicant: IRR, INC., Santa Barbara, CA (US)

(72) Inventor: Bruce Chandler May, Santa Barbara, CA (US)

(21) Appl. No.: 16/198,544

(22) Filed: Nov. 21, 2018

Related U.S. Application Data
(63) Continuation of application No. PCT/US2017/035426, filed on Jun. 1, 2017.
(60) Provisional application No. 62/345,435, filed on Jun. 3, 2016.

(51) Int. Cl. A61K 31/495; A61K 31/47; A61K 45/06; A61K 9/00

(52) U.S. Cl. CPC A61K 31/495; A61K 31/47; A61K 9/0019; A61K 9/0053; A61K 45/06

(57) ABSTRACT

Certain embodiments described herein include methods and formulations for treating or preventing symptoms and conditions associated with exposure to ionizing radiation and/or radiation generally. The methods and formulations include, but are not limited to, methods and formulations for delivering effective concentrations of levocetirizine and montelukast to a patient in need. The methods and formulations can comprise conventional and/or modified-release elements, providing for drug delivery to the patient.

### CROSS-REFERENCE TO RELATED APPLICATIONS

This patent application is a continuation of International Patent Application No. PCT/US2017/035426, filed Jun. 1, 2017, which claims the benefit of priority to U.S. Provisional Patent Application No. 62/345,435, filed Jun. 3, 2016. The foregoing applications are fully incorporated herein by reference for all purposes.

### BACKGROUND

### Field

The disclosure generally relates to treating patients exposed to radiation with the combination of levocetirizine and montelukast.

### Description of Related Art

Radiation can be emitted from sources such as the environment (e.g., from the sun, space, etc.), radioactive atoms (e.g., from bombs, medical isotopes, etc.), and/or from equipment (e.g., X-ray machines, CT scanners, etc.). Penetrating radiations include, for example, high energy X-rays, alpha particles, beta particles, gamma rays, high energy ultraviolet radiation, and neutrons. Radiation can cause damage to the tissues of living organisms and, in some circumstances, death.

### SUMMARY

A method of treating a patient exposed to radiation is disclosed. In some embodiments, the method comprises administering to a patient an effective amount of a combination of levocetirizine and montelukast.

In some embodiments, the method is useful for treating a patient exhibiting one or more complications selected from the group consisting of: pain, anorexia, nausea, vomiting, cramps, diarrhea, dehydration, electrolyte imbalance, fever, nervousness, burning sensations, confusion, headache, seizures, loss of consciousness, convulsions, coma, death of stem cells in the bone marrow, malaise, fatigue, drop in blood cell count, headache, fatigue, swelling, edema, cutaneous changes/damage (erythema, blistering, changes in pigmentation, dry and moist desquamation, ulceration, induration, fibrosis), loss of lymphocytes, blocking of cell maturation, altered cell signaling and/or trafficking, alterations in cellular differentiation and function, damage to immune/metabolic pathways, vascular injury, anemia, granulocytopenia, lymphocytopenia, thrombocytopenia, hemorrhage, loss of hematopoietic cells, bone marrow failure, infection, pneumonitis, acute respiratory distress, pulmonary fibrosis, acute and chronic renal injury, and renal failure. In some embodiments, the severity of one or more of those complications is reduced and/or the complication is alleviated completely, or substantially completely.

In some embodiments, the method involves the treatment of a patient exposed to radiation from one or more of fission-type nuclear bombs, thermonuclear bombs, “dirty bombs,” radiation therapy, medical radiation, space radiation (e.g., trapped radiation, galactic cosmic radiation (GCR), solar particle events (SPE)), cosmic rays, or high energy ultraviolet light.

In some embodiments, the combination of levocetirizine and montelukast is administered in a sequential manner.

In some embodiments, the combination of levocetirizine and montelukast is administered in a substantially simultaneous manner.

In some embodiments, the combination is administered to the patient by one or more of the routes consisting of enteral, intravenous, intraperitoneal, inhalation, intramuscular, subcutaneous and oral.

In some embodiments, the levocetirizine and montelukast are administered by the same route.

In some embodiments, the levocetirizine and montelukast are administered via different routes.

In some embodiments, one or more of levocetirizine or montelukast are provided as a slow release composition.

In some embodiments, the combination further comprises other medications known for use in treating complications associated with radiation exposure.

In some embodiments, the combination further comprises a steroid.

Some embodiments pertain to a method of treating a patient having a syndrome selected from the group consisting of Acute Radiation Syndrome, bone marrow syndrome (sometimes referred to as hematopoietic syndrome), gastrointestinal syndrome, pulmonary syndrome, cutaneous syndrome, cardiovascular syndrome/central nervous system (CNS) syndrome, the method comprising administering to the patient an effective amount of a combination of levocetirizine and montelukast.

Some embodiments provide a composition for use in treating a patient exposed to radiation, the composition comprising a combination of levocetirizine and montelukast.

### BRIEF DESCRIPTION OF THE DRAWINGS

FIG. 1 shows a diagram of a proposed anti-inflammatory mechanism of action of levocetirizine and montelukast utilizing a steroid model pathway.

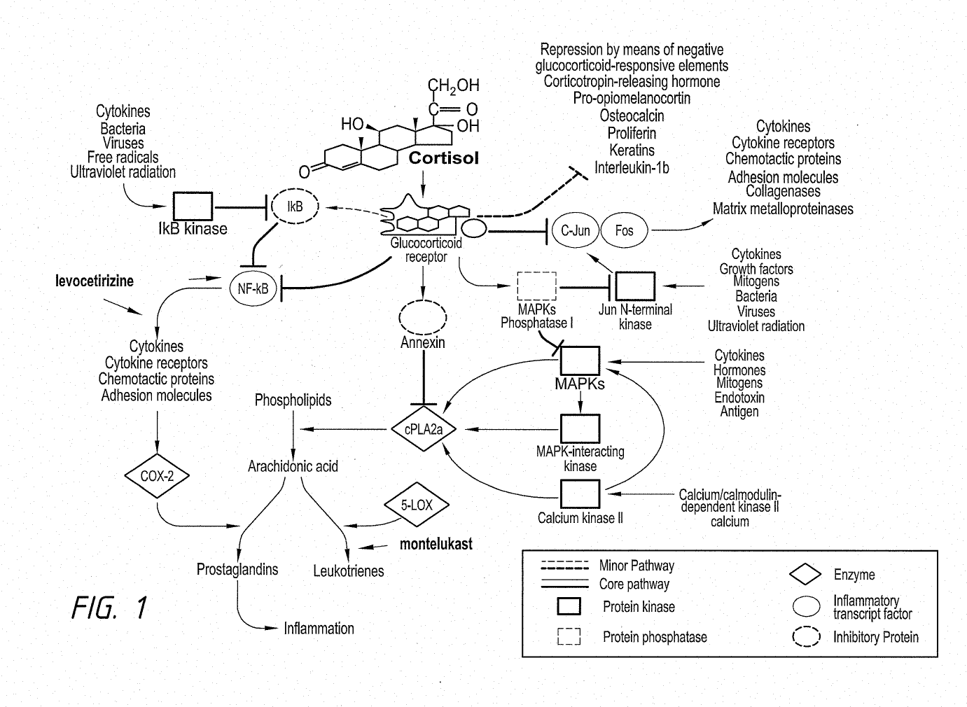

FIG. 2 depicts the clinical spectrum of radiation exposure. In some embodiments the dose of levocetirizine and montelukast may be titrated (e.g., increased or decreased) to correlate with the nature and extent of the radiation exposure.

### DETAILED DESCRIPTION

Some examples described herein illustrate the use of levocetirizine and montelukast as a medicament for the prevention or treatment of complications and damage caused by a subject's exposure to radiation. In some embodiments, subjects include those who are at risk of, who are being, or who have been exposed to radiation, including ionizing radiation. In some embodiments, radiation exposure can be treated where the radiation sources include, for example, fission-type nuclear bombs, thermonuclear bombs, radionuclides (e.g., used in radiation therapy for cancer treatment, radiative ablation, etc.), medical equipment (e.g., x-ray, PET, and CT machines), space (e.g., space radiation, trapped radiation, galactic cosmic radiation (GFR) and solar particle events (SPE)), or other types of radiation exposure. The examples described herein are illustrative and not intended in any way to restrict the general inventions presented and the various aspects and features of these inventions. Furthermore, the phraseology and terminology used herein is for the purpose of description and should not be regarded as limiting. No features or steps disclosed herein are essential or indispensable.

As used herein, “treat,” “treatment,” “treating,” “ameliorate,” “amelioration,” “ameliorating,” “improvement,” or “improving” refers to reducing, and/or alleviating the acute and/or long term effects of a radiation exposure. Treatment may comprise one or more of slowing progression, shortening duration, alleviating and/or reducing symptoms (or complications), alleviating and/or reducing associated secondary conditions, decreasing the duration of symptoms, decreasing the duration of associated secondary conditions, and/or alleviating or decreasing long term or residual effects and/or associated secondary issues. In some embodiments, “treating,” (or “treatment”) “ameliorating,” (or “ameliorate”) and/or “improving” (or “improvement”) refers to a detectable improvement and/or a detectable change consistent with improvement that occurs in a subject or in at least a minority of subjects, e.g., in at least about: 2%, 5%, 10%, 15%, 20%, 25%, 30%, 40%, 50%, 60%, 70%, 75%, 80%, 85%, 90%, 95%, 98%, 100%, or ranges including and/or spanning the aforementioned values. In some embodiments, “treating,” “ameliorating,” and/or “improving” refers to lower severity of symptoms associated with radiation exposure. In some embodiments such improvement or change may be observed in treated subjects as compared to subjects not treated with levocetirizine and montelukast, where the untreated subjects have been exposed to the same radiation source, or are subject to developing the same or similar disease condition, symptom, or the like. In some embodiments, treatment of a disease state, condition, symptom or assay parameter may be determined subjectively or objectively, e.g., self-assessment by a subject(s), by a clinician's assessment or by conducting an appropriate assay or measurement, including, e.g., a quality of life assessment, a slowed progression of a disease(s) or condition(s), a reduced severity of a disease(s) or condition(s), or a suitable assay(s) for the level or activity(ies) of a biomolecule(s), cell(s), by detection of respiratory or inflammatory disorders in a subject, and/or by modalities such as, but not limited to photographs, video, digital imaging, endoscopy, biopsy, and pulmonary function tests. Treatment may be transient, prolonged or permanent and/or it may be variable at relevant times during or after levocetirizine and montelukast are administered to a subject. Treatment with levocetirizine and montelukast may be evident from an assay (e.g., an in vitro assay, an in vivo assay, etc.). In some embodiments, the levocetirizine and montelukast treatment is curative. In some embodiments, the levocetirizine and montelukast combination successfully treats a patient when the combination is administered within timeframes described infra, or when administration occurs about 1 hour after, 1 day after, 1 week after, about 28 days after the subject(s) has been exposed to radiation. In some embodiments, the levocetirizine and montelukast treatment is preventative. In some embodiments, the levocetirizine and montelukast combination successfully treats a patient when the combination is administered within timeframes described infra, or when radiation exposure occurs about 1 hour after the administration or use of levocetirizine and montelukast to about 28 days, or 1, 3, 6, 9 months or more after a subject(s) has received such treatment.

The “modulation” of, e.g., a symptom or condition, level or biological activity of a molecule, or the like, refers, for example, to the symptom or activity, or the like that is detectably increased or decreased. Such increase or decrease may be observed in treated subjects as compared to subjects not treated with levocetirizine and montelukast, where the untreated subjects have, or are subject to developing, the same or similar disease state, condition, symptom or the like. Such increases or decreases may be at least about 2%, 5%, 10%, 15%, 20%, 25%, 30%, 40%, 50%, 60%, 70%, 75%, 80%, 85%, 90%, 95%, 98%, 100%, 150%, 200%, 250%, 300%, 400%, 500%, 1000% or more or ranges including and/or spanning the aforementioned values. Modulation may be determined subjectively or objectively, e.g., by the subject's self-assessment, by a clinician's assessment or by conducting an appropriate assay or measurement, including, e.g., quality of life assessments, suitable assays for the level or activity of molecules, cells or cell migration within a subject and/or by modalities such as, but not limited to photographs, video, digital imaging, endoscopy, biopsy, and pulmonary function tests. Modulation may be transient, prolonged or permanent or it may be variable at relevant times during or after levocetirizine and montelukast are administered to a subject or is used in an assay or other method described herein or a cited reference, e.g., within times described infra, or about 1 hour after the administration or use of levocetirizine and montelukast to about 3, 6, 9 months or more after a subject(s) has received levocetirizine and montelukast.

As used herein, the terms “prevent,” “preventing,” and “prevention” refer to the prevention of onset or development of damage associated with or caused by exposure to radiation. Preventing includes protecting against the occurrence and lowering the severity of damage associated with radiation.

As used herein, the terms “complications associated with radiation” include, but are not limited to, radiation sickness, acute radiation syndrome, radiation poisoning, radiation toxicity, acute damage to tissue caused by radiation, symptoms and secondary conditions associated with exposure to radiation (including, for example, pain, anorexia, nausea, vomiting, cramps, diarrhea, nervousness, confusion, loss of consciousness, burning sensations, convulsions coma, dehydration, electrolyte imbalance, death of stem cells in the bone marrow, fever, malaise, drop in blood cell count, headache, fatigue, skin irritation, erythema, pigmentation, dry and moist desquamation of the skin, fibrosis, necrosis, loss of lymphocytes, blocking of cell maturation, altered cell trafficking, alterations in cellular differentiation and function, damage to immune cell “niches,” hemorrhage, loss of hematopoietic cells, bone marrow failure, infection, pneumonitis, acute respiratory distress, pulmonary fibrosis, acute and chronic renal injury, renal failure, etc.), and death.

Some embodiments described herein provide a combination of levocetirizine and montelukast for the prevention, modulation, and/or treatment, of complications, symptoms, and/or other effects associated with exposure to radiation. In some embodiments, the radiation is an ionizing and/or penetrating radiation. Ionizing radiation is a type of radiation with enough energy to change atoms in tissues. Ionizing radiation can be emitted from sources such as radioactive atoms (e.g., from bombs or as medical isotopes) and/or from equipment (e.g., X-ray machines, CT scanners, etc.). Penetrating radiations include, for example, high energy X-rays, alpha particles, beta particles, gamma rays, positron radiation, high energy ultraviolet radiation, and neutrons. Exemplary radiation sources include fission-type nuclear bombs, thermonuclear bombs, “dirty bombs,” medical radioisotopes (e.g., for cancer treatment, radiative ablation, nuclear medicine, etc.), medical equipment (e.g., x-rays, PET), space radiation (e.g., trapped radiation, galactic cosmic radiation (GCR), solar particle events (SPE)), and the like. Space radiation consists primarily of ionizing radiation which exists in the form of high-energy, charged particles. Some embodiments described herein provide a combination of levocetirizine and montelukast for the prevention, modulation, and/or treatment, of complications, symptoms, and/or other effects associated with exposure to other forms of waves (e.g., nonelectromagnetic), including ultrasound waves, waves from sonic weaponry, compressions waves from explosive, etc. In some embodiments, the methods described herein pertain to treating complications associated with radiation from any one of these sources or other sources of radiation.

Radiation as described herein can include radiation from radionuclides. In some embodiments, the radionuclide is selected from the group consisting of barium-133, cadmium-109, cobalt-57, cobalt-60, europium-152, manganese-54, sodium-22, zinc-65, uranium-238, thorium, thallium-201, thallium-204, iridium-192, molybdenum-99, technetium-99m, tritium, polonium-210, caesium-137, americium-241, and the like. In some embodiments, the methods described herein pertain to treating or preventing complications associated with exposure to radiation from any one of these sources.

In addition to causing death in some circumstances, radiation exposure can result in a wide variety of symptoms and secondary conditions. The symptoms and secondary conditions accompanying acute exposure to ionizing radiation can include, but are not limited to: pain, anorexia, nausea, vomiting, cramps, diarrhea, dehydration, electrolyte imbalance, fever, burning sensations, nervousness, confusion, headache, loss of consciousness, seizures, coma, death of stem cells in the bone marrow, malaise, fatigue, swelling, edema, cutaneous changes/damage (erythema, blistering, changes in pigmentation, dry and moist desquamation, ulceration, induration, fibrosis), anemia, lymphocytopenia, granulocytopenia, thrombocytopenia, hemorrhage, bone marrow failure, blocking of cell maturation, altered cell trafficking, alterations in cellular differentiation and function, damage to immune/metabolic pathways, infection, pneumonitis, acute and chronic renal injury, and renal failure. These symptoms and secondary conditions are known as threshold effects and are dependent on the time of exposure, the time that has elapsed after radiation exposure, and amount of radiation exposure. Threshold effects may also include symptoms and secondary conditions including cataracts, sterility, hair loss, reduced thyroid function, development of autoimmune thyroid disease, pulmonary syndrome (ARDS—acute respiratory distress syndrome, pulmonary fibrosis), cardiovascular dysfunction/failure, and central nervous system dysfunction/failure. In some embodiments, the combination of levocetirizine and montelukast modulates, treats, or prevents one or more of these symptoms, conditions, or effects.

In some embodiments, the combination of levocetirizine and montelukast modulates, treats, or prevents threshold effects of radiation. In some embodiments, the combination of levocetirizine and montelukast modulates, treats, or prevents one or more of symptoms and secondary conditions associated with radiation exposure. For instance, in some embodiments, the combination of levocetirizine and montelukast modulates, treats, or prevents one or more of pain, anorexia, nausea, vomiting, cramps, diarrhea, dehydration, electrolyte imbalance, fever, nervousness, burning sensation, confusion, headache, seizures, loss of consciousness, coma, death of stem cells in the bone marrow, malaise, fatigue, swelling, edema, cutaneous changes/damage (erythema, blistering, changes in pigmentation, dry and moist desquamation, ulceration, induration, fibrosis), blocking of cell maturation, altered cell signaling/trafficking, alterations in cellular differentiation and function, damage to immune/metabolic pathways, vascular injury, anemia, granulocytopenia, lymphocytopenia, thrombocytopenia, hemorrhage, bone marrow failure, infection, pneumonitis, acute respiratory distress, pulmonary fibrosis, acute and chronic renal injury, and renal failure, cataracts, sterility, hair loss, and reduced thyroid function/development of autoimmune thyroid disease. In some embodiments, the combination of levocetirizine and montelukast reduces the risk and/or lessens the likelihood of a patient dying after radiation exposure.

Radiation may cause certain symptoms that overlap with those caused by other ailments, such as, allergy, cold, and/or flu (e.g., inflammatory skin conditions, etc.). In some embodiments, the combination of levocetirizine and montelukast modulates, treats, or prevents one or more of damage, symptoms, and/or associated secondary conditions associated with radiation exposure that overlap with symptoms caused by ailments, but where that damage, symptom, and/or associated secondary conditions are not caused by those other ailments. In some embodiments, the combination of levocetirizine and montelukast modulates, treats, or prevents one or more of damage, symptoms, and/or associated secondary conditions associated with radiation exposure that are not symptoms of allergy, cold, and/or flu.

In some embodiments, the combination of levocetirizine and montelukast modulates, treats, or prevents radiation damage, symptoms, and/or associated secondary conditions wherein the damage, symptoms, and/or associated secondary conditions are inflammation-mediated. In some embodiments, the combination of levocetirizine and montelukast modulates, treats, or prevents radiation damage, symptoms, and/or associated secondary conditions that are not skin conditions. In some embodiments, the combination of levocetirizine and montelukast modulates, treats, or prevents radiation damage, symptoms, and/or associated secondary conditions that are not inflammation-mediated and/or that are not inflammatory skin conditions. In some embodiments, the combination of levocetirizine and montelukast modulates, treats, or prevents radiation damage, symptoms, and/or associated secondary conditions that are not edemas or erythemas. In some embodiments, the combination of levocetirizine and montelukast modulates, treats, or prevents radiation damage, symptoms, and/or associated secondary conditions that are not IgE mediated. In some embodiments, the combination of levocetirizine and montelukast modulates, treats, or prevents inflammation-caused damage, symptoms, and/or associated secondary conditions wherein the inflammation is caused specifically by radiation (and not other sources of inflammation).

In some embodiments, the combination of levocetirizine and montelukast further comprises a steroid. In some embodiments, the combination of levocetirizine and montelukast does not include a steroid. In some embodiments, the combination of levocetirizine and montelukast does not include a corticotropin releasing factor (CRF).

In some embodiments, the combination of levocetirizine and montelukast modulates, treats, or prevents radiation damage, symptoms, and/or associated secondary conditions wherein the damage, symptoms, and/or associated secondary conditions are not associated with physical impact. For example, physical impact can include a crushing or penetrating injury such as from bodily injury sustained during a car accident, from a gunshot (e.g., a bullet penetrating tissue), or stab wound. In some embodiments, the combination of levocetirizine and montelukast modulates, treats, or prevents radiation damage, symptoms, and/or associated secondary conditions wherein the damage, symptoms, and/or associated secondary conditions are not associated with traumatic injury. In some embodiments, the combination of levocetirizine and montelukast modulates, treats, or prevents radiation damage, symptoms, and/or associated secondary conditions wherein the damage, symptoms, and/or associated secondary conditions are not associated with blood vessel disruption, occlusion, or rupture. For example, blood vessel disruption, occlusion, or rupture associated with stroke, embolism, aneurysm, etc.

Several acute syndromes are associated with exposure to ionizing radiation. One acute syndrome associated with exposure to ionizing radiation is known as Acute Radiation Syndrome (ARS) (i.e., radiation toxicity, radiation poisoning, or radiation sickness). ARS is an acute illness and is often caused by irradiation of the entire body (or most of the body) by a high dose of penetrating radiation in a very short period of time (usually a matter of minutes). The major cause of ARS is depletion of immature parenchymal stem cells in specific tissues. Those likely to suffer from ARS include but are not limited to people who are the survivors of the nuclear bombs (e.g., survivors of the Hiroshima and Nagasaki atomic bombs), those exposed to nuclear meltdowns (e.g., the firefighters that first responded after the Chernobyl Nuclear Power Plant event in 1986 or the Fukushima Daiichi meltdown in 2011), and those unintentionally exposures to sterilization irradiators. In some embodiments, the combination of levocetirizine and montelukast modulates, treats, or prevents one or more of the syndromes, symptoms, and secondary conditions as listed above and as disclosed elsewhere herein.

Syndromes associated with ARS can include: bone marrow syndrome (sometimes referred to as hematopoietic syndrome); gastrointestinal syndrome, pulmonary syndrome (ARDS—acute respiratory distress syndrome, pulmonary fibrosis), cutaneous syndrome, cardiovascular syndrome/central nervous system (CNS) syndrome. The symptoms and secondary conditions accompanying these syndromes can include in part: anorexia, nausea, vomiting, cramps, diarrhea, fatigue, shortness of breath, difficulty breathing, drop in O2 saturation, nervousness, confusion, loss of consciousness, burning sensations, seizures, coma, dehydration, electrolyte imbalance, death of stem cells in the bone marrow, fever, malaise, anemia, granulocytopenia, lymphocytopenia, and thrombocytopenia. In the skin, erythema is one of the first manifestations of skin damage followed by dry and moist desquamation, fibrosis, and necrosis. Each of these syndromes (and ARS generally) can result in death. In some embodiments, the combination of levocetirizine and montelukast modulates, treats, or prevents one or more of the syndromes, symptoms, and secondary conditions as listed above and as disclosed elsewhere herein.

The following table (Table 1) shows several classical presentations of ARS subsyndromes caused at various radiation dose thresholds.

TABLE 1

| | | Exposure to |
|---|---|---|
| Radiation Dose | Signs and Symptoms | Presentation, Time |
| 5 rem | Chromosome aberrations | 30 min |
| | first seen | |
| 12 rem | Reduction in sperm count | 42 ds |
| 0.75 Gy (75 rad) | Lymphocyte depletion | 6 h |
| 1 Gy (100 rad) | Nausea, vomiting | 6 h, then 5-7 d |
| 1-6 Gy (100-600 rad) | Hematopoietic syndrome | 1-6 h |
| 3 Gy (300 rad) | Temporary epilation | 14 d |
| 6 Gy (600 rad) | Erythema | 6-48 h, then 2-3 wk |
| | Pneumonitis | 4-6 wk |
| | Pulmonary syndrome | 1-6 mo |
| | (pulmonary fibrosis, | |
| | ARDS) | |
| 6-8 Gy (600-800 rad) | Gastrointestinal | 3-4 d |
| | syndrome | |
| 9-10 Gy (900-1000 rad) | Death | Days to weeks |
| >10 Gy (>1000 rad) | Neurovascular syndrome | Hours to days |

In some embodiments, the combination of levocetirizine and montelukast modulates, treats, or prevents one or more of the signs and symptoms of radiation exposure listed in Table 1 and as disclosed elsewhere herein.

In some embodiments, the combination of levocetirizine and montelukast modulates, treats, or prevents one or more of ARS (including one or more of the symptoms and/or secondary conditions related thereto), bone marrow syndrome, gastrointestinal syndrome, pulmonary syndrome (ARDS—acute respiratory distress syndrome, pulmonary fibrosis), cutaneous syndrome, cardiovascular syndrome/central nervous system (CNS) syndrome, or one or more symptoms thereof. In some embodiments, the combination of levocetirizine and montelukast prevents or decreases damage to parenchymal stem cells in patients exposed to radiation. In some embodiments, the combination of levocetirizine and montelukast modulates, treats, or prevents one or more symptoms and secondary conditions accompanying these syndromes, including, anorexia, nausea, vomiting, cramps, diarrhea, nervousness, confusion, loss of consciousness, burning sensations, convulsions coma, dehydration, electrolyte imbalance, death of stem cells in the bone marrow, fever, malaise, a drop in blood cell counts, and the like.

If protection from hematopoietic and gastrointestinal death can be achieved, the dose-limiting organ for survival is often the lung, with death related to delayed injuries such as pneumonitis. Two phases of functional injury to the lungs have been described in humans: an acute phase of radiation pneumonitis occurring 4-30 weeks after exposure and a later phase of fibrosis appearing 6-12 months after irradiation. Inflammation is the predominant early histological and physiological finding.

Radiation exposure may also cause non-threshold effects. Health effects of radiation that generally do not appear until years after an exposure are considered non-threshold. Any radiation exposure can increase a person's chances of having these effects. The chances of these delayed effects occurring increase with the size of the dose. They can occur in the person who receives the radiation dose or in that person's offspring (i.e., genetic effects). Non-threshold effects can include cancer (including second cancers), genetic effects, and fetal effects (embryo/fetus is particularly sensitive to radiation). Fetal effects could include mental disability and growth retardation. In some embodiments, the combination of levocetirizine and montelukast modulates, treats, or prevents one or more non-threshold effects (e.g., as described above). In some embodiments, the combination of levocetirizine and montelukast modulates, treats, or prevents one or more of cancer (including second cancers), genetic effects, and fetal effects.

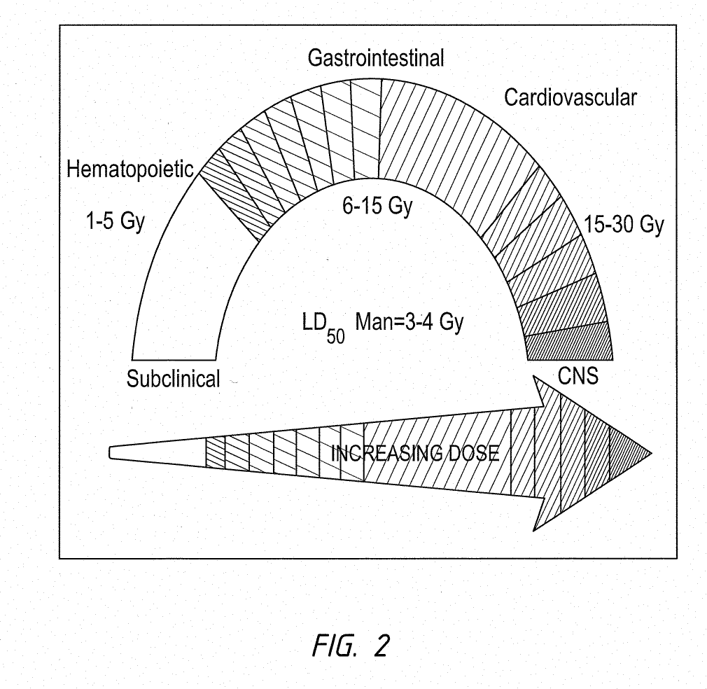

In some embodiments, the combination of levocetirizine and montelukast has protective effects against radiation of varying doses. In some embodiments, regardless of the amount of radiation exposure, a single dosage (e.g., a single dosing regimen) of the combination of levocetirizine and montelukast can be used. In some embodiments, because the combination of levocetirizine and montelukast is safe, equally high doses of the combination can be used in almost any situation. Alternatively, in some embodiments, as shown in FIG. 2, the dose of the combination of levocetirizine and montelukast may be titrated (up or down) to correlate with the nature and extent of the radiation exposure. In other words, in some embodiments, the dose of the combination of levocetirizine and montelukast is increased or decreased depending on the amount of radiation exposure the patient is exposed to. In some embodiments, the dose of the combination of levocetirizine and montelukast is not increased or decreased based on the amount of radiation exposure the patient is exposed to.

The amount of radiation can be quantified in numerous ways. The following provides several measures of radiation:

Rem: Roentgen Equivalent Man is a measurement of the biological effect of absorbed radiation.

Rad: Radiation absorbed dose is a measurement of the radiation absorbed by material or tissue.

Roentgen: Roentgen is the measurement of energy produced by gamma or X-ray radiation in a cubic centimeter of air.

For general purposes most physicists agree that Roentgen, Rad, and Rem may be considered equivalent.

System International (SI) Units: The SI units for radiation are ‘gray’ (Gy) and ‘sivert’ (Sv) for absorbed dose and equivalent dose respectively. The conversion from one system to another is as follows:

1 Sv=100 rem

1 mSv=100 mR (mrem)

1 Gy=100 rad; (100 rem)

1 mGy=100 mrad

As shown in FIG. 2, the Clinical Spectrum of Radiation Exposure estimates an LD (lethal dose) 50 at 60 days of approximately 400 rem (4 Gy).

In some embodiments, the combination of levocetirizine and montelukast has protective effects against radiation doses of at least about 0.5 rem, about 1 rem, about 10 rem, about 50 rem, about 60 rem, about 100 rem, about 200 rem, about 300 rem, about 400 rem, about 500 rem, about 1000 rem, about 5000 rem, about 10000 rem, or ranges including and/or spanning the aforementioned values. In some embodiments, the combination of levocetirizine and montelukast treats complications associated with radiation doses of at least about 0.5 rem, about 1 rem, about 10 rem, about 50 rem, about 60 rem, about 100 rem, about 200 rem, about 300 rem, about 400 rem, about 500 rem, about 1000 rem, about 5000 rem, about 10000 rem, or ranges including and/or spanning the aforementioned values. In some embodiments, the dose of the combination of levocetirizine and montelukast can be increased to correlate with the nature and extent of the radiation exposure.

The immune system responds rapidly and is quite sensitive to total body irradiation, in large part because of the tendency of lymphocytes to undergo apoptosis. Radiation-induced immune suppression and consequent infections are major consequences of ARS. Antibiotics can be administered in patient management as a countermeasure to infection. In some embodiments, the combination of levocetirizine and montelukast is able to boost and/or stabilize immune function to treat radiation induced immune suppression. In some embodiments, the combination can be used in conjunction with antibiotics to treat or prevent complications associated with radiation exposure.

Without being bound to any particular mechanism, it is believed that, in some embodiments, the combination of levocetirizine and montelukast treats patients with complications associated with radiation by virtue of the combination's ability to reduce inflammatory responses associated with radiation, thereby improving patients' clinical outcomes. The use of a combination of levocetirizine and montelukast targets multiple inflammatory pathways in the body, decreasing inflammation and allowing treatment of radiation sickness or alleviating complications associated with exposure to radiation. In some embodiments, the combination of levocetirizine and montelukast have a synergistic effect in the treatment of complications associated with radiation exposure. As described herein, in some embodiments, synergy between levocetirizine and montelukast shortens the course of radiation-related acute and chronic disease processes, thereby decreasing morbidity and mortality. In some embodiments, this combined therapy also can improve quality of life by the amelioration of symptoms/secondary conditions/side effects/disease process itself, and can decrease health-care costs.

Without being bound to any particular mechanism, it is believed that, in some embodiments, the combination of levocetirizine and montelukast treats patients with complications associated with radiation by virtue of certain anti-oxidative properties of the combination.

Levocetirizine is an antihistamine and montelukast is a leukotriene receptor antagonist. Levocetirizine, as a potent H1-antihistamine, acts in part by down-regulating the H1 receptor on the surface of mast cells and basophils to block the IgE-mediated release of histamine—the agent responsible for the cardinal symptoms of the innate immune response, including an inflammatory response, fever, sneezing, rhinorrhea, nasal congestion, itchy palate, and itchy red and watery eyes. Levocetirizine offers a short time to peak plasma level, 0.9 hr., a short time to steady state level, 40 hours, a low volume of distribution, 0.4 L/kg, and an enhanced receptor affinity of 5× over first generation mepyramine in an acidic pH (many acute inflammatory disease states are associated with acidosis, a low physiologic pH; increased 5×). Levocetirizine has a 24-hour receptor occupancy of ˜75%, the highest of the commercially available antihistamines. Receptor occupancy of the second generation antihistamines appears to correlate with the pharmacodynamic activity in skin wheal and flare studies and with efficacy in allergen challenge chamber studies. Levocetirizine is approved in the US for the treatment of perennial allergic rhinitis and chronic idiopathic urticaria down to six months of age. Levocetirizine is the most potent of the five modern generation antihistamines through histamine induced wheal and flare data. For example, levocetirizine at 5 mg per day is more effective than fexofenadine at its commonly prescribed dose of 180 mg per day in the United States. In Europe the adult dose is 120 mg per day.

Without being bound by a particular theory, the cellular mechanism of action for inflammatory reduction is a proposed reduction of the activation of the intracellular protein complex NF-κB (nuclear factor kappa B) which is in turn responsible for the reduction of I-CAM-1. I-CAM-1, a transmembrane protein, is viewed as the portal of entry of human rhinovirus into the cell. In addition, in some embodiments, levocetirizine decreases eosinophil migration and/or inflammatory mediators, IL-4, IL-6, and IL-8, IL-6, TNF-α (signaling proteins), regulating in part: fever and other bodily responses to trauma, and acute symptoms and complications caused by radiation exposure.

Montelukast, a leukotriene receptor antagonist, acts by binding with high affinity and selectivity to the CysLT1 receptor to inhibit the physiologic actions of the leukotriene LTD4. Leukotrienes are fatty signaling molecules whose effects include airway edema, smooth muscle contraction and altered cellular activity associated with the inflammatory process. Overproduction of leukotriene is a major cause of inflammation. The cysteinyl leukotrienes (LTC4, LTD4, LDE4) are products of arachidonic acid metabolism. These leukotrienes are released from various cells including mast cells and eosinophils. They bind to receptors in the human airway and on other pro-inflammatory cells including eosinophils and certain myeloid stem cells. Without being bound to any particular theory, it is thought that overproduction of leukotrienes contributes to inflammation associated with radiation exposure.

Montelukast is FDA approved in the US for the treatment of perennial allergic rhinitis, asthma, seasonal allergic rhinitis, and exercised induced bronchospasm. Montelukast is ineffective in improving asthma control or cold symptom scores caused by experimental rhinovirus infection. Analysis of secondary outcomes suggests that montelukast may protect against reductions in lung function and increases in sputum eosinophils caused by infections. During the recovery phase the percentage of sputum eosinophils was elevated in the placebo group, while the montelukast group remained at baseline levels. Further, peak expiratory flow was not decreased in the montelukast-treated patients. Montelukast treatment has no effect on the respiratory symptoms of patients with acute respiratory syncitial virus bronchiolitis.

Montelukast reaches a steady state level, like the second generation antihistamine, levocetirizine, in less than two days. Unlike other currently available leukotriene modulators, zileuton and zafirlukast, routine monitoring of liver function tests is not required. There are no drug interactions with warfarin, theophylline, digoxin, terfenadine, oral contraceptives, or prednisone.

Levocetirizine and montelukast are safe, i.e., FDA approved in the United States for allergic disorders down to age six months. They can be given primarily or in conjunction with many of the existing therapeutic protocols for the treatment of complications associated with radiation. In some embodiments, the combination of levocetirizine and montelukast can be administered for the treatment of radiation exposure or preventatively in patients, including pregnant women and children, that are under the age of about 1, about 2, about 3, about 4, about 5, about 10, about 15, or about 18. Moreover, both drugs have only once daily dosing, and no routine monitoring of blood work is necessary for most clinical situations. Further, both drugs exhibit minimal clinically relevant interactions with other medications. As described herein, both levocetirizine and montelukast reach steady state levels within two days to rapidly produce a synergistic and complementary anti-inflammatory effect.

Levocetirizine and montelukast are in different drug classes and target different receptors in the body. Because they target different receptors in the body, levocetirizine and montelukast can achieve their effect via different molecular pathways. In some embodiments, the combination of montelukast and levocetirizine achieves synergy to treat and/or provide a protective effect against radiation, either prior to, during, or after radiation exposure. In some embodiments, the synergistic effect shortens the course of complications caused by radiation and issues caused by radiation. In some embodiments, this synergistic effect is accomplished by the combination of levocetirizine and montelukast by targeting their respective different pathways in the body. In some embodiments, multiple inflammatory signaling pathways in the body are targeted to achieve protective effects or the treatment of radiation-based complications using levocetirizine and montelukast. In some embodiments, synergy is achieved by downregulating certain inflammatory processes. In some embodiments, the combination's effect to alleviate one or more disease states or symptoms associated with radiation exposure is achieved by stabilizing or reducing oxidative stress or physiological effects of oxidative stress caused by radiation. In some embodiments, synergy is achieved by enhancing certain antioxidant effects of the combination. In some embodiments, the use of the combination of montelukast and levocetirizine decreases one or more of the symptoms of, the duration of, morbidity from, and mortality from radiation-related disease states and symptoms. In some embodiments, the combination of levocetirizine and montelukast decreases the progression of complications associated with radiation exposure. In some embodiments, the combined levocetirizine and montelukast therapy can improve quality of life by ameliorating one or more of the symptoms, side effects, and the underlying radiation damage or complication itself, resulting in decreased health-care costs. In some embodiments, a synergistic effect can be observed in the use of a combination of levocetirizine and montelukast to treat inflammation.

Without being bound to any particular theory, it is believed that unchecked, pro-inflammatory reactions in the body can exacerbate biological effects and issues caused by radiation exposure. In some instances, these inflammatory responses contribute to the development and progression of complications associated with radiation exposure. In other instances, these inflammatory responses are themselves responsible for certain symptoms related to radiation exposure. In some embodiments, levocetirizine and montelukast act by down regulating pro-inflammatory mediators elicited by exposure to radiation, allowing the body to more readily react and recover from radiation exposure and complications associated with radiation exposure. In some embodiments, the levocetirizine and montelukast directly improve and/or resolve issues or symptoms caused by radiation exposure. Some embodiments provide the combination of levocetirizine and montelukast as a medicament for the treatment of complications associated with radiation that are exacerbated by or result from innate immune responses or adaptive immune responses caused by the radiation exposure.

Without being bound to any particular theory, the anti-inflammatory effect of the combination of levocetirizine and montelukast is due, at least in part, to the fact that both levocetirizine and montelukast affect eosinophil migration/quantity; the eosinophil is considered by scientists/clinicians as one hallmark of inflammation. Additionally, as discussed elsewhere herein, the response may be related, at least in part, due to levocetirizine's interference with the toll-like receptors (TLRs) and montelukast's separate interference with the leukotriene-related pathways to inflammation.

A common feature of all TLR recognition is the activation of three major signaling pathways: nuclear factor kappa-B (NF-κB), mitogen-activated protein kinase (MAPKs), and one or more of the interferon regulatory factors (IRFs). In some embodiments, the combination of levocetirizine and montelukast is used in methods to treat complications associated with radiation by blocking activation of one or more of these pathways. NF-κB plays a pivotal role across a spectrum of inflammation, immunity, cell proliferation, differentiation, and survival. NF-κB is expressed in almost all cell types and tissues. Specific binding sites are present in the promoters/enhancers of a large number of genes. For example, NF-κB target genes include: Cytokines/Chemokines and their Modulators, Immunoreceptors, Proteins Involved in Antigen Presentation, Cell Adhesion Molecules, Acute Phase Proteins, Stress Response Genes, Cell Surface Receptors, Regulators of Apoptosis, Growth Factors, Ligands and their Modulators, Early Response Genes, Transcription Factors and Regulators, Viruses, and Enzymes.

In some embodiments, the combination of levocetirizine and montelukast is used in methods to treat complications associated with radiation that elicit cellular activity or inflammatory responses via NF-κB. In some embodiments, the combination of levocetirizine and montelukast treats complications associated with radiation by blocking activation through the NF-κB pathway. In some embodiments, the combination of levocetirizine and montelukast treats complications associated with radiation by blocking TLR activation through the NF-κB pathway and at least one other cellular signaling pathway selected from the group consisting of the MAPKs pathway and the IRFs pathway. In some embodiments, the combination of levocetirizine and montelukast treats complications associated with radiation by blocking cellular signaling pathways other than those mediated by TLRs. In some embodiments, the combination of levocetirizine and montelukast reduces the activation of the NF-κB/toll-like receptors and/or other intracellular or extracellular protein complexes (e.g., exosomes, histones). In some embodiments, the combination of levocetirizine and montelukast treats complications associated with radiation that are activated at least in part through NF-κB.

One example of the influential nature the NF-κB family of transcription factors is RANTES (regulated on activation, normal T cell expressed and secreted). In the ‘late’ or adaptive phase of the immune response, RANTES is a chemokine generally expressed three to five days after T-cell activation. RANTES expression, mediated exclusively through NF-κB, attracts eosinophils, monocytes, mast cells and lymphocytes, activates basophils and induces histamine release from these cells. Select H1 receptor antagonists (e.g., levocetirizine) have the remarkable ability to inhibit NF-κB and activator protein-1 (AP-1) activity though H1 receptor—dependent and independent mechanisms.

Levocetirizine has been shown to inhibit human rhinovirus (HRV)-induced ICAM-1, cytokine expression, and viral replication in airway epithelial cells from both the nose and lung. Overexpression of the H1 receptor in the laboratory resulted in the inhibition of the HRV-induced upregulation of ICAM-1, 11-6, TLR3 expression and NF-κB activation. Levocetirizine reduced the levels of HRV-induced increases in ICAM-1 regardless of whether the levocetirizine was added before, after, or at the time of the HRV infection. The results were in agreement with previous research on the inhibitory effects of levocetirizine ICAM-1 up-regulation.

Without being bound to any particular theory, it is also believed that the combination of levocetirizine and montelukast may affect treatment of complications associated with radiation through anti-oxidative properties. For instance, it is believed that montelukast has antioxidant properties and that levocetirizine can enhance these anti-oxidative properties when administered concomitantly with montelukast (e.g., at a time when montelukast is present in the body). In some embodiments, the combination of levocetirine and montelukast acts as an antioxidative treatment for complications associated with radiation. In some embodiments, the combination of levocetirizine and montelukast acts synergistically as an antioxidative combination for the treatment or complications associated with radiation.

In some embodiments, the methods described herein involve identifying a patient (e.g., a subject) in need of treatment. In some embodiments, a patient may comprise any type of mammal (e.g., a mammal such as a human, cow, sheep, horse, cat, dog, goat, rodent, etc.). In some embodiments, patients in need of treatment include those who are at risk for being exposed to, that are being exposed to, or that have been exposed to radiation (e.g., by traveling to areas where radiation contamination is occurring or has occurred in the past). In some embodiments, patients in need of treatment can include those who are at a high likelihood of developing complications associated with radiation due to lifestyle variables (e.g., employees of power plants at nuclear facilities, etc.). In some embodiments, for at risk patient groups, the combination of levocetirizine and montelukast can be administered preventatively or curatively after about age: 1, 5, 10, 20, 30, 40, 50, 60, 70, 80, 90, or ranges including and/or spanning the aforementioned values, and throughout the rest of the patient's life.

Once identified as a patient, the combination of levocetirizine and montelukast is administered to the patient for a period of time. In some embodiments, the period of administration comprises a period starting prior to exposure to radiation, when the patient is first exposed to radiation, just after the patient is exposed to radiation, when the patient first displays symptoms, or when the patient has displayed symptoms for a period of more than about 1 day, about 2 days, about 3 days, about a week, about a month, or about two months. In some embodiments, the combination is administered until a time when the complications associated with radiation exposure are controlled or cured (e.g., the acute symptoms have subsided, symptoms have decreased to a baseline, risk factors for death have decreased, etc.), or for a prescribed period of time of less than about 1 week, about 2 weeks, about 3 weeks, about a month, about two months, about 6 months, or about a year. In some embodiments, the period of time comprises a period spanning from when the patient or an administrator of treatment (e.g., a doctor, nurse, medic, technician, relative, etc.) suspects the patient has been exposed to radiation to a time when the patient is no longer at risk of developing complications associated with radiation exposure. In some embodiments, the combination of levocetirizine and montelukast is given to alleviate symptoms of radiation exposure and the combination is given for the duration of the symptoms. In some embodiments, the combination of levocetirizine and montelukast is administered preventatively for a period during high exposure risk or during a period when the radiation exposure is likely to display symptoms.

Nuclear power plant workers exposed to radiation from a nuclear meltdown or others (e.g., astronauts, bomb survivors, etc.) can be administered the combination in an effective amount after radiation exposure in order to treat the worker. Alternatively, a nuclear meltdown clean-up crew can take the combination prophylactically (e.g., prior to starting clean-up) to prevent or modulate the effects of any potential radiation exposure. In some embodiments, an astronaut can take the combination before and/or during space travel to treat the effects of any exposure to space radiation, including trapped radiation, GFR and/or SPE (including radiation exposure from travel outside the Van Allen belt, travel outside Earth's magnetosphere, travel through the South Atlantic Anomaly, solar flares during space travel, magnetic storms encountered during space travel, etc.).

In some embodiments, dosing and delivery of the combination of levocetirizine and montelukast can be performed for periods between five days—twelve months to achieve continuous tissue levels of the drug combination. In some embodiments, dosing and delivery of levocetirizine and montelukast can be performed for periods of at least about: 1 day, 5 days, 10 days, 20 days, 30 days, 50 days, 100 days, 200 days, 300 days, or ranges including and/or spanning the aforementioned values. In some embodiments, the rationale is to achieve sustained tissue levels to modulate NF-κB at multiple targets within the immune system (Constant overexpression of the H1 Receptor).

In some embodiments, the levocetirizine montelukast combination is administered in a sequential manner. In some embodiments, levocetirizine is administered first. In some embodiments, montelukast is administered first. In some embodiments, the combination is administered in a substantially simultaneous manner.

In some embodiments, the combination is administered to the patient by one or more of the routes consisting of enteral, intravenous (including, but not limited to a long-acting injectable, e.g., an extended release preparation), intraperitoneal, inhalation, intramuscular (including, but not limited to a long-acting injectable, subcutaneous and oral). In some embodiments, the levocetirizine and montelukast are administered by the same route. In some embodiments, the levocetirizine and montelukast are administered by different routes. In some embodiments, the combination is dosed to the patient using an effective amount of a combination of levocetirizine and montelukast.

In some embodiments, levocetirizine and montelukast are provided in long-acting delivery formats to treat the complications associated with radiation exposure. In some embodiments, the long acting delivery formats deliver therapeutic doses of levocetirizine and montelukast for periods of at least about: 1 week, 2 weeks, 1 month, 6 months, or ranges including and/or spanning the aforementioned values. In some embodiments, the levocetirizine and montelukast are provided in once-daily or multiple-daily doses. In some embodiments, traditional oral delivery systems: film strips, bilayer tablets, capsules, tablets, nebulized therapy, etc. could be utilized if administered on at least a twice daily regimen, early in the course of the complication, i.e., the first seventy-two hours. Otherwise, with the onset of nausea and diarrhea, or manifestation of any of the systemic indicators: (a) shortness of breath, (b) hypotension, (c) bleeding, (d) coma, an IV (intravenous), IM (intramuscular) or LAI (long-acting injectable) can be successful in changing the outcome.

Depending upon the patient's age, weight, BMI (body mass index) and severity of the disease on presentation, the dosing (oral, IV, IM) or dose (LAI) can be titrated to effect over the following range:

Levocetirizine: 1.25 mg-30 mg/24 hours

Montelukast: 4 mg-50 mg/24 hours for a duration of at least five days

Computer modelling should allow for precise dosing and delivery which will vary depending upon the nature and extent of the clinical presentation. In some embodiments, the dose is adjusted depending on the patient's response to the combination or depending on the progression of the disease state.

In some embodiments, the typical daily dosage for levocetirizine is about 5 mg, about 10 mg, about 15 mg for adults. Studies in humans have shown that doses of levocetirizine up to 30 mg/day can be safely administered. In some embodiments, daily doses of levocetirizine can be at least about 1 mg, about 5 mg, about 10 mg, about 20 mg, about 30 mg, about 40 mg, about 50 mg, about 100 mg, about 500 mg, or ranges including and/or spanning the aforementioned values. Montelukast, a leukotriene receptor antagonist, acts concurrently to protect the respiratory tree as well as block mediators in the inflammatory cascade. The typical daily dosage of montelukast is 10 mg for adults. Montelukast has been administered at doses up to 200 mg/day to adult patients for 22 weeks and in short-term studies, and up to 900 mg/day to patients for approximately one week without clinically relevant side effects. In some embodiments, daily doses of montelukast can be at least about 1 mg, about 5 mg, about 10 mg, about 20 mg, about 30 mg, about 40 mg, about 50 mg, about 100 mg, about 200 mg, about 400 mg, about 600 mg, about 800 mg, about 1000 mg, about 2000 mg, about 4000 mg, about 6000 mg, or ranges including and/or spanning the aforementioned values.

In some embodiments, levels of levocetirizine utilized in the laboratory model can be safely achieved in a clinical setting; however, are above the standard adult dose of 5 mg daily used for the treatment of allergy and asthma. In some embodiments, the addition of montelukast, also above the standard 10 mg adult dose for allergy and asthma results in a remarkable synergistic effect which has been shown in our clinical setting to safely decrease the symptoms and duration of select viral infections (e.g., human rhinovirus, influenza).

Given the half-lives of the molecules and other pharmacokinetic considerations, once oral daily dosing, particularly in an acutely ill patients, may not be effective. As such, in some embodiments, in a difficult-to-treat or harsh environment, a long-acting injectable may be employed. In some embodiments, a formulation (e.g., a long-acting injectable) comprising 50-100 mg of levocetirizine and 100-200 mg of montelukast within a pharmaceutically acceptable medium or as a pharmaceutically acceptable medium (e.g., reconstituted lyophilized powder) is dosed to maintain a steady state level for seven days. In some embodiments, the injectable can be configured to deliver the oral equivalent of between 5 mg and 20 mg of levocetirizine and between 10 mg and 40 mg of montelukast to the patient per day (depending on the nature and extent of the disease process; taking into consideration patient weight, age, etc.). In some embodiments, oral dosing can also be used where appropriate dosing between 5 mg and 20 mg of levocetirizine and between 10 mg and 40 mg of montelukast/day, respectively. Divided oral daily dosing may be employed. In some embodiments, the formulation comprises about 50 mg, about 100 mg, about 150 mg, about 200 mg, about 300 mg, about 400 mg, about 500 mg, or more of levocetirizine. In some embodiments, the formulation comprises about 50 mg, about 100 mg, about 150 mg, about 200 mg, about 300 mg, about 400 mg, about 500 mg, or more of montelukast.

In some embodiments, long-acting comprises injectables that peak in a short period of time (e.g., within about 1-3 hours, or less than about 1 hour, about 2 hours, about 3 hours, about 4 hours, about 5 hours, or ranges including and/or spanning the aforementioned values). In some embodiments, long-acting injectables are those that maintain a nearly constant plasma or CNS level for a sustained period of time (e.g., at least about 3 days, about 4 days, about 5 days, about 6 days, about 7 days, about 14 days, about 21 days, about 28 days or more, or ranges including and/or spanning the aforementioned values). In some embodiments, a nearly constant blood concentration is one that is about 25 ng/mL (combined plateau of both drugs), about 50 ng/mL, about 150 ng/mL, about 250 ng/mL, about 350 ng/mL, about 450 ng/mL, about 550 ng/mL, about 650 ng/mL more than about 650 ng/mL, or ranges including and/or spanning the aforementioned values (plus or minus about 25-50 ng/mL).

The technology has evolved to repurpose levocetirizine+montelukast in a long-acting injectable. This concept is particularly useful: (a) where the patient is unable to swallow, (b) where the patient is unconscious, (c) where there are limited resources for overall care and management, (d) for prophylaxis in a time of war, (e) for use as a bioterrorist counteragent, and (f) during travel in space.

Predictive modelling software can be utilized to take existing information on the API (active pharmaceutical ingredient), excipients, the desired release profile, and end environment (body v CNS) and calculate a formulation which can then be used to manufacture microparticles that encapsulate the API and release it at the desired rate. Using computer metrics, the laboratory to manufacturing formulation variances can be minimized during the design phase.

Delivery vehicles include but are not limited to injectable microparticles, nanoparticles, matrix implants, and device coatings. Release profiles can be designed as constant rate (where doses are released at desired profiles for a period of days, weeks, or months), delayed release, or sequential release. In some embodiments, a wide variety of controlled release systems can be formulated. In some embodiments, the delivery vehicle is selected from the group consisting of injectable microparticle, nanoparticles, pellets, rods discs, tablets, thin film coatings, matrix implants, device coatings, and combinations thereof. In some embodiments, the delivery vehicle formulated from one or more of Poly(lactic-co-glycolic acid) (PLGA), Polyanhydrides (PSA, PSA:FAD), Polylactides (PLA), Poly-ortho-esters (POE), or HPMC hydrogels. The release profile can be tailored between Constant Rate (days, weeks, months), Delayed Release, and Sequential Release.

Without being bound to a particular theory, delivery of levocetirizine and montelukast (e.g., sustained, intermittent, or otherwise) will stabilize NF-κB through the overexpression of the H1-receptor in a dose-dependent manner.

In some embodiments, oral BID dosing can be used to saturate levocetirizine and montelukast receptors in an estimated ratio of 3 mg/6 mg (respectively) one in the AM and two HS. Separately, 6 mg/12 mg at night for long-term treatment. In some embodiments, where therapy would be long-term, months to years, qd to bid with an optimal daily dosing range of 6-9 mg/12-18 mg: levo/monte; titrated to effect as determined from monthly to quarterly patient visits, neuropsychiatric assessments at six month intervals and QOL questionnaires at each patient visit. In some embodiments, both molecules cross the blood-brain barrier at 0.1 mg/kg. In some embodiments, lower (or higher) dosing could be used.

In some embodiments, the combination of levocetirizine and montelukast can be given instead of, or in conjunction with, existing therapeutic protocols for the treatment of, including but not limited to, complications associated with radiation therapy or exposure. Levocetirizine and montelukast can also be given instead of, or in conjunction with, existing therapeutic protocols for inflammation related to, including but not limited to, complications associated with radiation therapy or exposure.

### EXAMPLES

### Example 1: Atom Bomb

Based on the inventor's clinical experience using levocetirizine and montelukast, the following results are projected using controlled studies.

A cohort of 40 patients between the ages of 15-30 years of age exposed to nuclear radiation from the detonation of an atomic bomb is identified. Each patient is identified as having a symptom of ARS (e.g., one or more of pain, anorexia, nausea, vomiting, cramps, diarrhea, nervousness, confusion, loss of consciousness, burning sensations, seizures, coma, dehydration, electrolyte imbalance, fever, malaise, drop in hemoglobin and platelet counts, headache, fatigue, cutaneous changes, drop in lymphocyte and/or granulocyte counts, blocking of cell maturation (low reticulocyte count), bone marrow failure (evidenced by a bone marrow biopsy, as required), hemorrhage, infection, pneumonitis, acute and chronic renal injury, renal failure, etc.), ideally within the first 48 hours of symptom onset. The experimental group patients (n=20) receives levocetirizine and montelukast. The control group patients (n=20; “CONT”) receive conventional treatments for radiation exposure.

Age, sex, race, height, weight, BMI (Body Mass Index/kg/m²), vital signs, O₂ saturation on room air, major medical problems, medications, allergies to medications, habits, social history, previous surgery, and photographs of the patient are logged at the initial visit and the patient's symptom profile tracked in a controlled environment.

Distance from ground zero and the duration of exposure are logged for each patient following the blast.

Dosimetry is performed if available.

If possible, an applicable quality of life questionnaire is filled out by the patient and health care provider.

Onset, duration, and intensity of symptoms are logged in addition to the time to resolution of symptoms (time zero—first dose of medication(s)).

Depending upon the treatment location, the following studies and special chemistries may not be available. If available, the following testing is performed.

Screening laboratory/imaging studies consisting of a complete blood count, comprehensive metabolic panel, C-reactive protein, Sed rate, T and B cell lymphocyte panel, PT, PTT, D-dimer, reticulocyte count, chest x-ray, CT Scan of the Chest, Face, Brain, and Abdomen as indicated by the nature and extent of the radiation/blast exposure, EKG, viral and bacterial cultures, blood cultures and aerobic cultures of the airway are taken the time of presentation.

Additional specimens are drawn for analysis/or stored for additional research;

Once to twice daily serum levels of levocetirizine and montelukast for seven days;

Samples for chemokines, cytokines, biomarkers of inflammation, and biomarkers of coagulopathy. These include but are not limited to: Granulocyte macrophage colony stimulating factor (GM-CSF); GROα; Interferon α2 (IFNα2); IFNβ; IFNγ; IL-10; Interleukin 12p70 (IL-12p70); IL12p40; Interleukin 1α (IL-1α); IL-1β; IL-1 receptor antagonist (IL-1RA); IL-2; IL-4; IL-5; IL-6; IL-8; IFN-γ-inducible protein 10 (IP-10); Monocyte chemoattractant protein 1 (MCP-1); Macrophage colony stimulating factor (MCSF); MIP-α; MIP-1β; Soluble CD40 ligand (sCD40L); Soluble E-selectin (sE-selectin); Soluble Fas ligand (sFasL); Tumor necrosis factor α (TNF-α); Vascular endothelial growth factor A (VEGF-A); D-dimer; Tissue plasminogen activator (TPA); Plasminogen activator inhibitor-1 (PAI-1); Serum amyloid antigen (SAA); Regulated on activation, normal T-cell expressed and secreted (RANTES); sVCAM-1; Fibrinogen; Ferritin; Cortisol; Tissue factor (TF); Thrombomodulin; S100B protein; Cellular prion protein (PrP^{C}); Ubiquitin C-terminal hydrolase-L1 (UCH-L1); choline (cell membrane damage); Myo-inositol (cell membrane damage or reactive astrogliosis); Tau protein; p-Tau (phosphorylated Tau); ICAM-1 (Intercellular adhesion molecule 1); ICAM-5 (Intercellular adhesion molecule 5); GFAP (Glial fibrillary acidic protein); NRGN (Neurogranin); SNCB (Beta-Synuclein); MT3 (Metallothionein 3); and injury specific exosomes/microRNA.

### Dosing

In some embodiments, in the treatment of life-threatening complications associated with radiation exposure, sustained tissue levels are used to oversaturate the H1 and leukotriene receptors in order to achieve the desired clinical outcome. The above markers are used to analyze patient response and further define the mechanism of action of the medication. In some embodiments, one or more of the above cytokines, chemokines, biomarkers of inflammation, and biomarkers for coagulopathy return to normal/non-exposed levels at an accelerated rate versus CONT groups.

For levocetirizine, peak concentrations are typically 270 ng/ml and 308 ng/ml following a single and repeated 5 mg once daily oral dose, respectively.

For montelukast, the pharmacokinetics are nearly linear for oral doses up to 50 mg with safety studies up to 900 mg/day for one week. A standard 10 mg oral dose is reflected by a mean AUC 2689 ng/hr/ml (range 1521 to 4595) and mean Cmax of 353 ng/ml.

Given the half-lives of the molecules and other pharmacokinetic considerations, once daily oral dosing, particularly in an acutely ill patient with nausea and vomiting, may not be effective. As such, particularly in a difficult-to-treat environment, a long-acting injectable may be employed. For instance, a long acting injectable comprising 50-100 mg of levocetirizine and 100-200 mg of montelukast within a pharmaceutically acceptable medium (e.g., reconstituted lyophilized powder) is dosed to maintain a steady state level for seven days. The injectable can be configured to deliver the oral equivalent of between 5 mg and 20 mg of levocetirizine and between 10 mg and 40 mg of montelukast to the patient per day (depending on the nature and extent of the disease process; taking into consideration patient weight, age, etc.). Oral dosing can also be used where appropriate to achieve similar blood levels (e.g., daily, bid, tid, or more).

With a high mortality rate depending on the radiation dose, it would be considered unethical to conduct a placebo arm. As such, 10 EXPT patients would receive a long-acting injectable preparation (computer modeled on a mg/kg basis) to deliver a sustained plasma level of both levocetirizine and montelukast in the range of 350 ng/ml for 7 days (the EXPT1 group). The second group of 10 patients would receive a higher dose to sustain plasma levels in the range of 500 ng/ml for a period of 7 days (The EXPT2 group), while the CONT group receives conventional radiation treatments. The EXPT1 and EXPT2 groups also receive conventional radiation supportive measures in addition to the levocetirizine and montelukast combination.

### Outcomes

The primary endpoint of the study is the percentage reduction in mortality and symptoms when compared to age and symptom matched controls at time zero. The long-acting injectable is found to decrease in the mortality based on published mortality rates. A dose-response curve trends favorably to the higher mean serum concentration range of 500 ng/ml.

Patients receiving the levocetirizine and montelukast exhibit statistically significantly improved outcomes relative to the CONT group. The death rate for the EXPT1 and EXPT2 groups at day 14 is 25% and 30% lower, respectively, than the CONT group. Of the survivors, those in the EXPT1 and EXPT2 groups have 30% and 40% lower incidences of prolonged secondary issues resulting from the acute exposure, respectively. Severity of pain decrease by 15% and 25% in the EXPT1 and EXPT2 groups, respectively, compared to the CONT group. Severity of anorexia decreases by 25% and 45% in the EXPT1 and EXPT2 groups, respectively, compared to the CONT group. Severity of nausea decreases by 29% and 42% in the EXPT1 and EXPT2 groups, respectively, compared to the CONT group. Severity of vomiting decreases by 48% and 62% in the EXPT1 and EXPT2 groups, respectively, compared to the CONT group. Severity of cramps decreases by 24% and 46% in the EXPT1 and EXPT2 groups, respectively, compared to the CONT group. Severity of diarrhea decreases by 20% and 45% in the EXPT1 and EXPT2 groups, respectively, compared to the CONT group. Severity of nervousness decreases by 30% and 55% in the EXPT1 and EXPT2 groups, respectively, compared to the CONT group. Severity of confusion decreases by 20% and 45% in the EXPT1 and EXPT2 groups, respectively, compared to the CONT group. Severity of duration of losses of consciousness decreases by 28% and 56% in the EXPT1 and EXPT2 groups, respectively, compared to the CONT group. Severity of the acute cutaneous changes (erythema, dry and moist desquamation and ulceration) decreases by 10% and 25% in the EXPT1 and EXPT2 groups, respectively, compared to the CONT group. Severity of seizures decreases by 38% and 52% in the EXPT1 and EXPT2 groups, respectively, compared to the CONT group. Number of comas decrease by 80% and 90% in the EXPT1 and EXPT2 groups, respectively, compared to the CONT group. Severity of dehydration decreases by 20% and 43% in the EXPT1 and EXPT2 groups, respectively, compared to the CONT group. Severity of electrolyte imbalance decreases by 22% and 46% in the EXPT1 and EXPT2 groups, respectively, compared to the CONT group. The degree of stem cell death in the bone marrow decreases by 20% and 35% in the EXPT1 and EXPT2 groups, respectively, compared to the CONT group. Fever decreases by 2° F. and 3° F. in the EXPT1 and EXPT2 groups, respectively, compared to the CONT group. Severity of malaise decreases by 22% and 46% in the EXPT1 and EXPT2 groups, respectively, compared to the CONT group. Red blood cell counts increase by 20% and 30% in the EXPT1 and EXPT2 groups, respectively, compared to the CONT group; platelet counts correspondingly increase. Severity of headache decreases by 23% and 40% in the EXPT1 and EXPT2 groups, respectively, compared to the CONT group. Severity of fatigue decreases by 21% and 48% in the EXPT1 and EXPT2 groups, respectively, compared to the CONT group. Lymphocyte and granulocyte counts increase in the range of 28% and 45% respectively, in the EXPT1 and EXPT2 groups, respectively, compared to the CONT group. Severity of hemorrhage decreases by 18% and 38% in the EXPT1 and EXPT2 groups, respectively, compared to the CONT group. The incidence of bone marrow failure decreases by 20% and 30% in the EXPT1 and EXPT2 groups, respectively, compared to the CONT group. The incidence of renal failure decrease by 25% and 35% in the EXPT1 and EXPT2 groups, respectively, compared to the CONT group. The incidence of infection decreases proportionately with the increase in granulocyte counts in the EXPT1 and EXPT2 groups, respectively, compared to the CONT group. Incidences of pneumonitis decrease by 45% and 55% in the EXPT1 and EXPT2 groups, respectively, compared to the CONT group.

### Example 2: Nuclear Power Plant Radiation Exposure

Based on the inventor's clinical experience using levocetirizine and montelukast, the following results are projected using controlled studies.

A cohort of 40 patients between the ages of 25-45 years of age exposed to nuclear radiation from the meltdown of a nuclear reactor is identified. Each patient is identified as having one or more symptoms of ARS selected from pain, nausea, vomiting, cramps, diarrhea, loss of consciousness, burning sensations, electrolyte imbalance, death of stem cells in the bone marrow, fever, drop in red blood cell and platelet counts, headache, fatigue, cutaneous changes, drop in lymphocyte and/or granulocyte counts, blocking of cell maturation (low reticulocyte count), bone marrow failure (evidenced by a bone marrow biopsy, as required), infection, pneumonitis, or anorexia, ideally within the first 48 hours of symptom onset. The experimental group patients (n=20) receives levocetirizine and montelukast. The control group patients (n=20; “CONT”) receive conventional treatments for radiation exposure.

Age, sex, race, height, weight, BMI (Body Mass Index/kg/m²), vital signs, O₂ saturation on room air, major medical problems, medications, allergies to medications, habits, prior surgery, and photographs of the patient are logged at the initial visit and the patient's symptom profile tracked in a controlled environment.

Dosimetry is performed if available.

If possible, an applicable quality of life questionnaire is filled out by the patient and health care provider.

Onset, duration, and intensity of symptoms are logged in addition to the time to resolution of symptoms (time zero—first dose of medication(s)).

Depending upon the treatment location, the following studies and special chemistries may not be available. If available, the following testing is performed.

Screening laboratory/imaging studies consisting of a complete blood count, comprehensive metabolic panel, C-reactive protein, Sed rate, T and B cell lymphocyte panel, PT, PTT, D-dimer, reticulocyte count, chest x-ray, CT Scan of the Chest, Abdomen, and Brain as indicated by the nature and extent of the radiation exposure, EKG, viral and bacterial cultures, blood cultures and aerobic cultures of the airway are taken the time of presentation.

Additional specimens are drawn for analysis/or stored for additional research;

Once to twice daily serum levels of levocetirizine and montelukast for seven days;

Samples for chemokines, cytokines, biomarkers of inflammation, and biomarkers of coagulopathy. These include but are not limited to: Granulocyte macrophage colony stimulating factor (GM-CSF); GROα; Interferon α2 (IFNα2); IFNβ; IFNγ; IL-10; Interleukin 12p70 (IL-12p70); IL12p40; Interleukin 1α (IL-1α); IL-10; IL-1 receptor antagonist (IL-1RA); IL-2; IL-4; IL-5; IL-6; IL-8; IFN-γ-inducible protein 10 (IP-10); Monocyte chemoattractant protein 1 (MCP-1); Macrophage colony stimulating factor (MCSF); MIP-α; MIP-1β; Soluble CD40 ligand (sCD40L); Soluble E-selectin (sE-selectin); Soluble Fas ligand (sFasL); Tumor necrosis factor α (TNF-α); Vascular endothelial growth factor A (VEGF-A); D-dimer; Tissue plasminogen activator (TPA); Plasminogen activator inhibitor-1 (PAI-1); Serum amyloid antigen (SAA); Regulated on activation, normal T-cell expressed and secreted (RANTES); sVCAM-1; Fibrinogen; Ferritin; Cortisol; Tissue factor (TF); Thrombomodulin; S100B protein; Cellular prion protein (PrP^{C}); Ubiquitin C-terminal hydrolase-L1 (UCH-L1); choline (cell membrane damage); Myo-inositol (cell membrane damage or reactive astrogliosis); Tau protein; p-Tau (phosphorylated Tau); ICAM-1 (Intercellular adhesion molecule 1); ICAM-5 (Intercellular adhesion molecule 5); GFAP (Glial fibrillary acidic protein); NRGN (Neurogranin); SNCB (Beta-Synuclein); MT3 (Metallothionein 3); and injury specific exosomes/microRNA.

### Dosing

For levocetirizine, peak concentrations are typically 270 ng/ml and 308 ng/ml following a single and repeated 5 mg once daily oral dose, respectively.

For montelukast, the pharmacokinetics are nearly linear for oral doses up to 50 mg with safety studies up to 900 mg/day for one week. A standard 10 mg oral dose is reflected by a mean AUC 2689 ng/hr/ml (range 1521 to 4595) and mean Cmax of 353 ng/ml.

Given the half-lives of the molecules and other pharmacokinetic considerations, once daily oral dosing, particularly in an acutely ill patient with nausea and vomiting, may not be effective. As such, particularly in a difficult-to-treat environment, a long-acting injectable may be employed. For instance, a long acting injectable comprising 50-100 mg of levocetirizine and 100-200 mg of montelukast within a pharmaceutically acceptable medium (e.g., reconstituted lyophilized powder) is dosed to maintain a steady state level for seven days. The injectable can be configured to deliver the oral equivalent of between 5 mg and 20 mg of levocetirizine and between 10 mg and 40 mg of montelukast to the patient per day (depending on the nature and extent of the disease process; taking into consideration patient weight, age, etc.). Oral dosing can also be used where appropriate to achieve similar blood levels (e.g., daily, bid, tid, or more).

With a high mortality rate depending on the radiation dose, it would be considered unethical to conduct a placebo arm. As such, 10 experimental group patients would receive a long-acting injectable preparation (computer modeled on a mg/kg basis) to deliver a sustained plasma level of both levocetirizine and montelukast in the range of 350 ng/ml for 7 days (the EXPT1 group). The second group of 10 patients would receive a higher dose to sustain plasma levels in the range of 500 ng/ml for a period of 7 days (The EXPT2 group), while the CONT group receives conventional radiation treatments. The EXPT1 and EXPT2 groups also receive conventional radiation supportive measures in addition to the levocetirizine and montelukast combination.

### Outcomes

The primary endpoint of the study is the percentage reduction in mortality and symptoms when compared to age and symptom matched controls at time zero. The long-acting injectable is found to decrease in the mortality by up to 55% based on published mortality rates. A dose-response curve trends favorably to the high mean serum concentration range of 500 ng/ml.

Patients receiving the levocetirizine and montelukast exhibit statistically significantly improved outcomes relative to the CONT group. The death rate for the EXPT1 and EXPT2 groups at day 14 is 30% and 52% lower, respectively, than the CONT group. Of the survivors, severity of pain decreases by 25% and 35% in the EXPT1 and EXPT2 groups, respectively, compared to the CONT group. Severity of nausea decreases by 39% and 48% in the EXPT1 and EXPT2 groups, respectively, compared to the CONT group. Severity of vomiting decreases by 52% and 72% in the EXPT1 and EXPT2 groups, respectively, compared to the CONT group. Severity of cramps decreases by 44% and 58% in the EXPT1 and EXPT2 groups, respectively, compared to the CONT group. Severity of diarrhea decreases by 30% and 45% in the EXPT1 and EXPT2 groups, respectively, compared to the CONT group. Duration of losses of consciousness decrease by 33% and 54% in the EXPT1 and EXPT2 groups, respectively, compared to the CONT group. Severity of acute cutaneous changes (erythema, dry and moist desquamation, and ulceration) decreases by 22% and 38% in the EXPT1 and EXPT2 groups, respectively, compared to the CONT group. Severity of electrolyte imbalance decreases by 25% and 49% in the EXPT1 and EXPT2 groups, respectively, compared to the CONT group. The degree of stem cell death in the bone marrow decreases by 20% and 35% in the EXPT1 and EXPT2 groups, respectively, compared to the CONT group Fever decreases by 2° F. and 3° F. in the EXPT1 and EXPT2 groups, respectively, compared to the CONT group. Red blood cell count are 22% and 37% higher in the EXPT1 and EXPT2 groups, respectively, compared to the CONT group; platelet counts correspondingly increase. Severity of headache decreases by 28% and 46% in the EXPT1 and EXPT2 groups, respectively, compared to the CONT group. Severity of fatigue decreases by 28% and 43% in the EXPT1 and EXPT2 groups, respectively, compared to the CONT group. Lymphocyte and granulocyte counts increase in the range of 30% and 39%, respectively in the EXPT1 and EXPT2 groups, respectively, compared to the CONT group. Hemorrhage severity is 26% and 47% lower in the EXPT1 and EXPT2 groups, respectively, compared to the CONT group. The incidence of bone marrow failure decreases by 20% and 30% in the EXPT1 and EXPT2 groups, respectively, compared to the CONT group. Incidences of infection decrease by 72% and 81% in the EXPT1 and EXPT2 groups, respectively, compared to the CONT group. Incidences of pneumonitis decrease by 45% and 55% in the EXPT1 and EXPT2 groups, respectively, compared to the CONT group. Incidences of pneumonitis decrease by 45% and 55% in the EXPT1 and EXPT2 groups, respectively, compared to the CONT group. Severity of anorexia decreases by 25% and 45% in EXPT1 and EXPT2 groups respectively, compared to the CONT group.

### Example 3: Medical Radiation

Based on the inventor's clinical experience using levocetirizine and montelukast, the following results are projected using controlled studies.

A cohort of 40 patients between the ages of 25-65 years of age exposed to radiation from a radionuclide during cancer treatment is identified. Each patient is identified as having one or more symptoms selected from pain, nausea, vomiting, cramps, diarrhea, loss of hair, and anorexia, ideally within the first 48 hours of symptom onset. The experimental group patients (n=20; “EXPT”) receives levocetirizine and montelukast. The control group patients (n=20; “CONT”) receive conventional treatments for radiation exposure.

Age, sex, race, height, weight, BMI (Body Mass Index/kg/m²), vital signs, O₂ saturation on room air, major medical problems, medications, allergies to medications, habits, prior surgery, and photographs of the patient are logged at the initial visit and the patient's symptom profile tracked in a controlled environment.

Dosimetry is performed if available.

If possible, an applicable quality of life questionnaire is filled out by the patient and health care provider.

Onset, duration, and intensity of symptoms are logged in addition to the time to resolution of symptoms (time zero—first dose of medication(s)).

Depending upon the treatment location, the following studies and special chemistries may not be available. If available, the following testing is performed.

Screening laboratory studies consisting of a complete blood count, comprehensive metabolic panel, C-reactive protein, Sed rate, T and B cell lymphocyte panel, PT, PTT, D-dimer, chest x-ray, EKG, viral and bacterial cultures, blood cultures and aerobic cultures of the airway are taken the time of presentation.

Additional specimens are drawn for analysis/or stored for additional research:

Once to twice daily serum levels of levocetirizine and montelukast for seven days;

### Dosing

Levocetirizine 5-20 mg, divided dose bid, or in an injectable format to achieve sustained serum levels of at least 350 ng/ml plus

Montelukast 10-40 mg/day, divided dose bid, or in an injectable format to achieve sustained dosing of at least 350 ng/ml.

### Outcomes

Patients receiving the levocetirizine and montelukast exhibit statistically significantly improved outcomes relative to the CONT group. Pain decreases by 35% in the EXPT group compared to the CONT group. Severity of nausea decreases by 38% in the EXPT compared to the CONT group. Severity of vomiting decreases by 55% in EXPT compared to the CONT group. Severity of cramps decreases by 34% in EXPT group compared to the CONT group. Severity of diarrhea decreases by 34% in EXPT group compared to the CONT group. Hair loss decreases by 25% in the EXPT group compared to the CONT group. Anorexia resolves in the EXPT group compared to the CONT group.

### Example 4: Hydrogen Bomb

Based on the inventor's clinical experience using levocetirizine and montelukast, the following results are projected using controlled studies.

A cohort of 40 patients between the ages of 5-65 years of age exposed to nuclear radiation from the detonation of a hydrogen bomb is identified. Each patient is identified as having one or more symptoms of ARS selected from pain, nausea, vomiting, cramps, diarrhea, death of stem cells in the bone marrow, fever, drop in red blood cell and platelet counts, headache, fatigue, hemorrhage, drop in lymphocyte and/or granulocyte counts, cutaneous changes, or anorexia, ideally within the first 48 hours of symptom onset.

Distance from ground zero and the duration of exposure are logged for each patient following the blast.

Dosimetry is performed if available.

The experimental group patients (n=20) receives levocetirizine and montelukast. The control group patients (n=20; “CONT”) receive conventional treatments for radiation exposure.

Age, sex, race, height, weight, BMI (Body Mass Index/kg/m²), vital signs, O₂ saturation on room air, major medical problems, medications, allergies to medications, habits, prior surgery, and photographs of the patient are logged at the initial visit and the patient's symptom profile tracked in a controlled environment.

If possible, an applicable quality of life questionnaire is filled out by the patient and health care provider.

Onset, duration, and intensity of symptoms are logged in addition to the time to resolution of symptoms (time zero—first dose of medication(s)).

Depending upon the treatment location, the following studies and special chemistries may not be available. If available, the following testing is performed.

Screening laboratory/imaging studies consisting of a complete blood count, comprehensive metabolic panel, C-reactive protein, Sed rate, T and B cell lymphocyte panel, PT, PTT, D-dimer, chest x-ray, CT Scan of the Chest, Abdomen, and Brain as indicated by the nature and extent of the radiation exposure, EKG, viral and bacterial cultures, blood cultures and aerobic cultures of the airway are taken the time of presentation.

Additional specimens are drawn for analysis/or stored for additional research;

Once to twice daily serum levels of levocetirizine and montelukast for seven days;

Samples for chemokines, cytokines, biomarkers of inflammation, and biomarkers of coagulopathy. These include but are not limited to: Granulocyte macrophage colony stimulating factor (GM-CSF); GROα; Interferon α2 (IFNα2); IFNβ; IFNγ; IL-10; Interleukin 12p70 (IL-12p70); IL12p40; Interleukin 1α (IL-1α); IL-1β; IL-1 receptor antagonist (IL-1RA); IL-2; IL-4; IL-5; IL-6; IL-8; IFN-γ-inducible protein 10 (IP-10); Monocyte chemoattractant protein 1 (MCP-1); Macrophage colony stimulating factor (MCSF); MIP-α; MIP-10; Soluble CD40 ligand (sCD40L); Soluble E-selectin (sE-selectin); Soluble Fas ligand (sFasL); Tumor necrosis factor α (TNF-α); Vascular endothelial growth factor A (VEGF-A); D-dimer; Tissue plasminogen activator (TPA); Plasminogen activator inhibitor-1 (PAI-1); Serum amyloid antigen (SAA); Regulated on activation, normal T-cell expressed and secreted (RANTES); sVCAM-1; Fibrinogen; Ferritin; Cortisol; Tissue factor (TF); Thrombomodulin; S100B protein; Cellular prion protein (PrP^{C}); Ubiquitin C-terminal hydrolase-L1 (UCH-L1); choline (cell membrane damage); Myo-inositol (cell membrane damage or reactive astrogliosis); Tau protein; p-Tau (phosphorylated Tau); ICAM-1 (Intercellular adhesion molecule 1); ICAM-5 (Intercellular adhesion molecule 5); GFAP (Glial fibrillary acidic protein); NRGN (Neurogranin); SNCB (Beta-Synuclein); MT3 (Metallothionein 3); and injury specific exosomes/microRNA.

### Dosing

For levocetirizine, peak concentrations are typically 270 ng/ml and 308 ng/ml following a single and repeated 5 mg once daily oral dose, respectively. In extreme cases this can be doubled, tripled, otherwise increased. In mild cases, this amount can be halved, quartered, or otherwise reduced.

For montelukast, the pharmacokinetics are nearly linear for oral doses up to 50 mg with safety studies up to 900 mg/day for one week. In extreme cases this can be doubled, tripled, otherwise increased. In mild cases, this amount can be halved, quartered, or otherwise reduced. A standard 10 mg oral dose is reflected by a mean AUC 2689 ng/hr/ml (range 1521 to 4595) and mean Cmax of 353 ng/ml.

With a high mortality rate depending on the radiation dose, it would be considered unethical to conduct a placebo arm. As such, 10 experimental group patients would receive a long-acting injectable preparation (computer modeled on a mg/kg basis) to deliver a sustained plasma level of both levocetirizine and montelukast in the range of 350 ng/ml for 7 days (the EXPT1 group). The second group of 10 patients would receive a higher dose to sustain plasma levels in the range of 500 ng/ml for a period of 7 days (The EXPT2 group), while the CONT group receives conventional radiation treatments. The EXPT1 and EXPT2 groups also receive conventional radiation supportive measures in addition to the levocetirizine and montelukast combination.

### Outcomes

The primary endpoint of the study is the percentage reduction in mortality. The secondary endpoint is the reduction in symptoms when compared to age and symptom matched controls at time zero. The long-acting injectable is found to decrease in the mortality by up to 68% based on published mortality rates. A dose-response curve trends favorably to the high mean serum concentration range of 500 ng/ml.

Patients receiving the levocetirizine and montelukast exhibit statistically significantly improved outcomes relative to the CONT group. The death rate for the EXPT1 and EXPT2 groups at day 14 is 30% and 52% lower, respectively, than the CONT group. Of the survivors, severity of pain decreases by 28% and 38% in the EXPT1 and EXPT2 groups, respectively, compared to the CONT group. Severity of nausea decreases by 32% and 48% in the EXPT1 and EXPT2 groups, respectively, compared to the CONT group. Severity of vomiting decreases by 53% and 70% in the EXPT1 and EXPT2 groups, respectively, compared to the CONT group. Severity of cramps decreases by 41% and 59% in the EXPT1 and EXPT2 groups, respectively, compared to the CONT group. Severity of diarrhea decreases by 31% and 45% in the EXPT1 and EXPT2 groups, respectively, compared to the CONT group. The degree of stem cell death in the bone marrow decreases by 20% and 35% in the EXPT1 and EXPT2 groups, respectively, compared to the CONT group Fever decreases by 2° F. and 3° F. in the EXPT1 and EXPT2 groups, respectively, compared to the CONT group. Red blood cell count are 25% and 33% higher in the EXPT1 and EXPT2 groups, respectively, compared to the CONT group; platelet counts correspondingly increase. Severity of headache decreases by 22% and 46% in the EXPT1 and EXPT2 groups, respectively, compared to the CONT group. Severity of fatigue decreases by 38% and 53% in the EXPT1 and EXPT2 groups, respectively, compared to the CONT group. Hemorrhage severity is 36% and 57% lower in the EXPT1 and EXPT2 groups, respectively, compared to the CONT group. Lymphocyte and granulocyte counts increase in the range of 30% and 38%, respectively, in the EXPT1 and EXPT2 groups, respectively, compared to the CONT group. Severity of the acute cutaneous manifestations (erythema, dry and moist desquamation and ulceration) decreases by 28% and 48% in the EXPT1 and EXPT2 groups respectively, compared to the CONT group. Severity of anorexia decreases by 25% and 45% in the EXPT1 and EXPT2 groups, respectively, compared to the CONT group.

### Example 5: Dirty Bomb

Based on the inventor's clinical experience using levocetirizine and montelukast, the following results are projected using controlled studies.

A cohort of 40 patients between the ages of 5-65 years of age exposed to nuclear radiation from a dirty bomb is identified. Each patient is identified as having one or more symptoms of ARS selected from pain, nausea, vomiting, cramps, diarrhea, death of stem cells in the bone marrow, fever, drop in red blood cell and platelet counts, headache, fatigue, hemorrhage, drop in lymphocyte and/or granulocyte counts, or cutaneous changes, ideally within the first 48 hours of symptom onset. The experimental group patients (n=20) receives levocetirizine and montelukast. The control group patients (n=20; “CONT”) receive conventional treatments for radiation exposure.

Age, sex, race, height, weight, BMI (Body Mass Index/kg/m²), vital signs, O₂ saturation on room air, major medical problems, medications, allergies to medications, habits, prior surgery, and photographs of the patient are logged at the initial visit and the patient's symptom profile tracked in a controlled environment.

Distance from ground zero and the duration of exposure are logged for each patient following the blast.

Dosimetry is performed if available.

If possible, an applicable quality of life questionnaire is filled out by the patient and health care provider.

Onset, duration, and intensity of symptoms are logged in addition to the time to resolution of symptoms (time zero—first dose of medication(s)).

Depending upon the treatment location, the following studies and special chemistries may not be available. If available, the following testing is performed.

Screening laboratory/imaging studies consisting of a complete blood count, comprehensive metabolic panel, C-reactive protein, Sed rate, T and B cell lymphocyte panel, PT, PTT, D-Dimer, chest x-ray, CT Scan of the Chest, Face, Brain and Abdomen as indicated by the nature and extent of the radiation/blast exposure, EKG, viral and bacterial cultures, blood cultures and aerobic cultures of the airway are taken the time of presentation.

Additional specimens are drawn for analysis/or stored for further analysis:

Once to twice daily serum levels of levocetirizine and montelukast for seven days; Dosing

For levocetirizine, peak concentrations are typically 270 ng/ml and 308 ng/ml following a single and repeated 5 mg once daily oral dose, respectively. In extreme cases this can be doubled, tripled, otherwise increased. In mild cases, this amount can be halved, quartered, or otherwise reduced.

For montelukast, the pharmacokinetics are nearly linear for oral doses up to 50 mg with safety studies up to 900 mg/day for one week. In extreme cases this can be doubled, tripled, otherwise increased. In mild cases, this amount can be halved, quartered, or otherwise reduced. A standard 10 mg oral dose is reflected by a mean AUC 2689 ng/hr/ml (range 1521 to 4595) and mean Cmax of 353 ng/ml.

With a high mortality rate depending on the radiation dose, it would be considered unethical to conduct a placebo arm. As such, 10 experimental group patients would receive a long-acting injectable preparation (computer modeled on a mg/kg basis) to deliver a sustained plasma level of both levocetirizine and montelukast in the range of 350 ng/ml for 7 days (the EXPT1 group). The second group of 10 patients would receive a higher dose to sustain plasma levels in the range of 500 ng/ml for a period of 7 days (The EXPT2 group), while the CONT group receives conventional radiation treatments. The EXPT1 and EXPT2 groups also receive conventional radiation supportive measures in addition to the levocetirizine and montelukast combination.

### Outcomes

The primary endpoint of the study is the percentage reduction in mortality. The secondary endpoint is the reduction in symptoms when compared to age and symptom matched controls at time zero. The long-acting injectable is found to decrease in the mortality by up to 42% based on published mortality rates. A dose-response curve trends favorably to the high mean serum concentration range of 500 ng/ml.

Patients receiving the levocetirizine and montelukast exhibit statistically significantly improved outcomes relative to the CONT group. The death rate for the EXPT1 and EXPT2 groups at day 14 is 38% and 54% lower, respectively, than the CONT group. Of the survivors, severity of pain decreases by 26% and 42% in the EXPT1 and EXPT2 groups, respectively, compared to the CONT group. Severity of nausea decreases by 33% and 47% in the EXPT1 and EXPT2 groups, respectively, compared to the CONT group. Severity of vomiting decreases by 43% and 65% in the EXPT1 and EXPT2 groups, respectively, compared to the CONT group. Severity of cramps decreases by 41% and 59% in the EXPT1 and EXPT2 groups, respectively, compared to the CONT group. Severity of diarrhea decreases by 31% and 45% in the EXPT1 and EXPT2 groups, respectively, compared to the CONT group. The degree of stem cell death in the bone marrow decreases by 25% and 33% in the EXPT1 and EXPT2 groups, respectively, compared to the CONT group Fever decreases by 2° F. and 3° F. in the EXPT1 and EXPT2 groups, respectively, compared to the CONT group. Red blood cell count are 28% and 32% higher in the EXPT1 and EXPT2 groups, respectively, compared to the CONT group; platelet counts correspondingly increase. Severity of headache decreases by 22% and 46% in the EXPT1 and EXPT2 groups, respectively, compared to the CONT group. Severity of fatigue decreases by 38% and 53% in the EXPT1 and EXPT2 groups, respectively, compared to the CONT group. Hemorrhage severity is 38% and 42% lower in the EXPT1 and EXPT2 groups, respectively, compared to the CONT group. Severity of the acute cutaneous manifestations (erythema, dry and moist desquamation, and ulceration) deceases by 28% and 38% in the EXPT1 and EXPT2 groups respectively, compared to the CONT group.

### Example 6: Space Travel

Based on the inventor's clinical experience using levocetirizine and montelukast, the following results are projected using controlled studies.

Ten astronauts between the ages of 34 and 45 years of age are identified before a prolonged space mission. The experimental group patients (n=5) receives levocetirizine and montelukast. The control group patients (n=5; “CONT”) receive no treatment.

Age, sex, race, height, weight, BMI (Body Mass Index/kg/m²), vital signs, major medical problems, medications, allergies to medications, cigarette and alcohol use, social history, and previous surgery are logged at the initial visit and the patient's symptom profile tracked in a controlled environment.

A global quality of life questionnaire is filled out by the patient and health care provider. Screening laboratory/imaging studies include of a complete blood count, comprehensive metabolic panel, C-reactive protein, T and B cell lymphocyte panel, Chest CT Scan, functional and molecular MRIs of the Brain, EKG, viral and bacterial cultures, blood cultures and aerobic cultures of the airway are taken.

Additional specimens are drawn for analysis/or stored for additional research;

Weekly serum levels of levocetirizine and montelukast for six months;

Samples for chemokines, cytokines, biomarkers of inflammation, and biomarkers of coagulopathy are drawn weekly for six months. These include but are not limited to: Granulocyte macrophage colony stimulating factor (GM-CSF); GROα; Interferon α2 (IFNα2); IFNβ; IFNγ; IL-10; Interleukin 12p70 (IL-12p70); IL12p40; Interleukin 1α (IL-1α); IL-1β; IL-1 receptor antagonist (IL-1RA); IL-2; IL-4; IL-5; IL-6; IL-8; IFN-γ-inducible protein 10 (IP-10); Monocyte chemoattractant protein 1 (MCP-1); Macrophage colony stimulating factor (MCSF); MIP-α; MIP-1β; Soluble CD40 ligand (sCD40L); Soluble E-selectin (sE-selectin); Soluble Fas ligand (sFasL); Tumor necrosis factor α (TNF-α); Vascular endothelial growth factor A (VEGF-A); D-dimer; Tissue plasminogen activator (TPA); Plasminogen activator inhibitor-1 (PAI-1); Serum amyloid antigen (SAA); Regulated on activation, normal T-cell expressed and secreted (RANTES); sVCAM-1; Fibrinogen; Ferritin; Cortisol; Tissue factor (TF); Thrombomodulin; S100B protein; Cellular prion protein (PrP^{C}); Ubiquitin C-terminal hydrolase-L1 (UCH-L1); choline (cell membrane damage); Myo-inositol (cell membrane damage or reactive astrogliosis); Tau protein; p-Tau (phosphorylated Tau); ICAM-1 (Intercellular adhesion molecule 1); ICAM-5 (Intercellular adhesion molecule 5); GFAP (Glial fibrillary acidic protein); NRGN (Neurogranin); SNCB (Beta-Synuclein); MT3 (Metallothionein 3); and injury specific exosomes/microRNA.

### Dosing

In some embodiments, in the treatment (e.g., the prevention) of symptoms and or the biological effects associated with space radiation exposure, sustained tissue levels are used to oversaturate the H1 and leukotriene receptors in order to achieve the desired clinical outcome. The above markers are used to analyze patient response and further define the mechanism of action of the medication. In some embodiments, one or more of the above cytokines, chemokines, biomarkers of inflammation, and biomarkers for coagulopathy remain at normal/non-exposed levels relative to CONT groups.

For levocetirizine, peak concentrations are typically 270 ng/ml and 308 ng/ml following a single and repeated 5 mg once daily oral dose, respectively. In some embodiments, because the radiation exposure is less than that anticipated for example, a nuclear bomb or dirty bomb, 2.5 mg, i.e., half a standard 5 mg tablet can be administered during extended space travel.

For montelukast, the pharmacokinetics are nearly linear for oral doses up to 50 mg with safety studies up to 900 mg/day for one week. A standard 10 mg oral dose is reflected by a mean AUC 2689 ng/hr/ml (range 1521 to 4595) and mean Cmax of 353 ng/ml. In some embodiments, because the radiation exposure is less than that anticipated for example, a nuclear bomb or dirty bomb, 5 mg, i.e., half a standard 10 mg tablet can be administered during extended space travel.

In a difficult-to-treat environment such as space, a long-acting injectable may be employed. For instance, a long acting injectable comprising 50-100 mg of levocetirizine and 100-200 mg of montelukast within a pharmaceutically acceptable medium (e.g., reconstituted lyophilized powder) is dosed to maintain a steady state level for up to two months or longer. The injectable can be configured to deliver the oral equivalent of between 5 mg and 20 mg of levocetirizine and between 10 mg and 40 mg of montelukast to the patient per day (depending on the nature and extent of the disease process; taking into consideration patient weight, age, etc.). Oral dosing can also be used where appropriate to achieve similar blood levels (e.g., daily, bid, tid, or more). In some embodiments, because the radiation exposure is somewhat lower than radiation exposure from, for instance, a nuclear bomb, approximately half-dosing ranges can be utilized.

### Outcomes

The primary endpoint of the study is the percentage reduction in symptoms and biological markers when compared to matched controls at time zero. The long-acting injectable is found to decrease symptoms and biomakers based on comparison to the CONT group. Astronauts receiving the levocetirizine and montelukast exhibit statistically significantly improved global quality of life outcomes relative to the CONT group. The EXPT astronauts using the combination of levocetirizine and montelukast have similar biomarker levels when compare to time zero (prior to space travel). The CONT group however, show higher biomarker levels (statistically significant) corresponding to their cumulative space travel radiation exposure.

### Example 7: Rat Study

Based on the inventor's clinical experience using levocetirizine and montelukast, the following results are projected using controlled studies.

Animal Model: WAG/Rij female rats

Preliminary Design: two arms

(1) control 12 rats

(2) treatment 12 rats/allometric scaling—levocetirizine+montelukast

Length of Study: 12 weeks

Radiation Dose: 13 Gy to the whole thorax (rate: 1.43 Gy/min); LD80/8 weeks

Analysis: Survival (primary end-point), necropsy, pleural fluid volume if present at necropsy, breathing rate

Pilot project design (for companion follow-up rat and primate studies): 7-day (single dose) long acting injectable (LAI) administered for 12 weeks/Rat dose adjusted (scaled) based on a mean rat weight—compared to an average human weight). Ultimate delivery can be optimized as an LAI from 24 hours—two months.

### Outcomes

The long-acting injectable is found to increase survival rates by 75% versus the control group.

### Example 8: Rat Study

Based on the inventor's clinical experience using levocetirizine and montelukast, the following results are projected using controlled studies.

Levocetirizine and montelukast as a countermeasure to radiation exposure is tested in a rat lung model prior to nonhuman primate studies and use in humans. Using the rat lung as the target to dominate the response, an in vivo dose response curve for normal lung tissue can be calculated, i.e., is a known and can be validated as a very steep sigmoid curve, increasing rapidly above a threshold.

Dose Modifying Factor (DMF)—measure of effectiveness of therapy as a radiation countermeasure

To measure the effectiveness of the combination therapy, the DMF or dose modifying factor is measured. This parameter is the dose required for a given effect in the experimental (WAG/Rij rat) group (EXPR) divided by the dose to achieve the same effect in the control group (CONTR). As a point of reference, the generally accepted DMF as an effective radioprotector or mitigator is in excess of 1.15.

As alternative indices of protection, an agent is considered for further study in larger animal models such as dogs or nonhuman primates when the countermeasure in the rat:

1. Shifts the dose-response curve to higher radiation doses by at least 1 Gy, or

2. Increases in the duration of survival at the LD80/10 (80% mortality at 10 weeks) by at least 50%.

Outcome: WAG/Rij Rat Model—12 weeks/Initial Radiation Dose—13 Gy to the Whole Thorax

Within the discrete parameters of the study: 13 Gy to the whole thorax (rate: 1.43 Gy/min); LD80/8 weeks, the combination of levocetirizine and montelukast allometrically scaled and delivered weekly as a long-acting injectable, will shift the dose-response curve by at least one Gy/increase the duration of survival by at least 50%/yield a DMF in excess of 1.15.

What is claimed is:

1. A method of treating a patient exposed to radiation, the method comprising administering to the patient an effective amount of a combination of levocetirizine and montelukast.

2. The method of claim 1, wherein the treatment causes a decrease in severity or instances of one or more complications selected from the group consisting of pain, anorexia, nausea, vomiting, cramps, diarrhea, dehydration, electrolyte imbalance, fever, nervousness, burning sensations, confusion, headache, seizures, loss of consciousness, coma, death of stem cells in the bone marrow, malaise, fatigue, swelling, edema, cutaneous changes/damage (erythema, blistering, changes in pigmentation, dry and moist desquamation, ulceration, induration, fibrosis), blocking of cell maturation, altered cell signaling/trafficking, alterations in cellular differentiation and function, damage to immune/metabolic pathways, vascular injury, anemia, granulocytopenia, lymphocytopenia, thrombocytopenia, hemorrhage, bone marrow failure, infection, pneumonitis, acute respiratory distress, pulmonary fibrosis, acute and chronic renal injury, and renal failure.

3. The method of claim 1, wherein the radiation exposure is from one or more of a fission-type nuclear bomb, thermonuclear bomb, a “dirty bomb,” radiation therapy, medical radiation, space radiation (e.g., trapped radiation, galactic cosmic radiation (GCR), solar particle events (SPE)), or high energy ultraviolet light.

4. The method of claim 1, wherein the combination of levocetirizine and montelukast is administered in a sequential manner.

5. The method of claim 1, wherein the combination of levocetirizine and montelukast is administered in a substantially simultaneous manner.

6. The method of claim 1, wherein the combination is administered to the patient by one or more of the routes consisting of enteral, intravenous, intraperitoneal, inhalation, intramuscular, subcutaneous and oral.

7. The method of claim 1, wherein the levocetirizine and montelukast are administered by the same route.

8. The method of claim 1, wherein the levocetirizine and montelukast are administered via different routes.

9. The method of claim 1, wherein one or more of levocetirizine or montelukast are provided as a slow release composition.

10. The method of claim 1, wherein the combination further comprises other medications known for use in treating complications associated with radiation exposure.

11. The method of claim 1, wherein the combination further comprises a steroid.

12. A method of treating a patient having a syndrome selected from the group consisting of Acute Radiation Syndrome, bone marrow syndrome (sometimes referred to as hematopoietic syndrome), gastrointestinal syndrome, pulmonary syndrome, cutaneous syndrome, and cardiovascular/central nervous system (CNS) syndrome, the method comprising administering to the patient an effective amount of a combination of levocetirizine and montelukast.

13. A composition for use in treating a patient exposed to radiation, the composition comprising a combination of levocetirizine and montelukast.
